(19) United States
(12) Patent Application Publication
(10) Pub. No.: US 2014/0005148 A1
(43) Pub. Date: Jan. 2, 2014

(54) STABLE LIQUID FORMULATIONS OF NITROGEN MUSTARDS

(71) Applicant: Coldstream Laboratories Inc., Lexington, KY (US)

(72) Inventors: Sundar Neelakantan, Lexington, KY (US); Eric W. Smart, Nicholasville, KY (US); Charles Reichel, Georgetown, KY (US)

(21) Appl. No.: 13/929,963

(22) Filed: Jun. 28, 2013

Related U.S. Application Data
(60) Provisional application No. 61/666,228, filed on Jun. 29, 2012.

(51) Int. Cl. A61K 31/675; A61K 31/198; A61K 31/196

(52) U.S. Cl. CPC A61K 31/675; A61K 31/196; A61K 31/198
USPC 514/90; 514/567

(57) ABSTRACT

Described are stable liquid formulations of nitrogen mustards. The nitrogen mustard includes mechlorethamine, cyclophosphamide, ifosfamide, melphalan, chlorambucil, uramustine, thiotepa and combinations thereof. The formulation includes a non-aqueous liquid having at least a first solvent either individually or in combination with one or more additional solvents. The formulation further includes an antioxidant, an organic acid, and a source of chloride ions.

### RELATED APPLICATION

This application claims the benefit of and priority to Provisional Application Ser. No. 61/666,228 filed Jun. 29, 2012, the disclosure of which is expressly incorporated herein by reference in its entirety.

### FIELD

The present invention relates generally to liquid formulations of nitrogen mustards and more particularly to stable liquid formulations of nitrogen mustards.

### BACKGROUND

Nitrogen mustards are a class of DNA alkylating oncology agents that are administered by injection. Nitrogen mustards exhibit poor stability in aqueous solutions due to their rapid degradation. Thus, most commercial parenteral nitrogen mustard preparations are manufactured as dry powders. Such powders are typically produced by lyophilization. The lyophilized solid products are generally reconstituted with water or other suitable diluent immediately prior to administration. However, manufacturing and administering solid forms of injectable drugs present several problems.

For example, the time and complexity associated with lyophilization adds significantly to the overall manufacturing costs for the drug. The most common and desirable lyophilization solvent is water. The reactivity of most nitrogen mustards in aqueous solution presents a challenge to the industrial scale manufacture of lyophilized products. In certain instances, organic solvents can be used to dissolve nitrogen mustards and these organic solutions can then be lyophilized. However, the use of organic solvents in lyophilization presents safety issues with respect to residual solvent content in the finished product and in the safe handling of such materials during manufacturing.

Lyophilized powders or cakes are more difficult to inspect for particulate contamination because foreign matter may be hidden by the drug itself (as compared to clear solutions which are relatively easy to inspect either visually or by means of an automated inspection process).

Lyophilized powders and cakes are more difficult to sterilize as compared to liquid solutions. While lyophilized products can be sterile-filtered prior to lyophilization, certain drugs are manufactured by filling powdered ingredients directly to vials. In such cases, the formulation components must be sterile prior to initiation of the production of the finished drug product or terminally sterilized by exposure to heat or ionizing radiation. The inherent instability of nitrogen mustards renders them generally difficult to terminally sterilize. Inherent limitations in most pharmaceutical chemical plants render it difficult to ensure sterility and that the materials are free from extraneous particulate matter.

Precisely controlling the amount of product distributed in the form of powders and cakes can be more difficult as compared to liquid solutions. Powder filling operations are inherently imprecise due to the difficulties in handling small amounts of single doses of a powdered drug. However, liquid dispensing equipment can easily and precisely administer single doses of liquid formulations.

Administration of freeze-dried products is potentially complex. For example, the healthcare provider administering lyophilized pharmaceutical products must carefully follow the reconstitution instructions in order to achieve the desired concentration of the drug. The procedures vary from product to product but can involve calculation and careful measurement of a diluent followed by agitation of the product to achieve a homogenous solution of the product. Once reconstituted, the liquid must be carefully inspected by the healthcare provider to ensure that the product is completely dissolved and free of visible particulate contamination. This visual check is the only practical opportunity to observe the fully dissolved product prior to administration to the patient (as opposed to 100% visual inspection of a liquid product following manufacture at the manufacturing facility). Many drugs that require reconstitution exhibit poor solution stability and must be administered to the patient promptly after reconstitution. In the event of a delay between reconstitution and administration that exceeds the limit specified in the package insert, the product must be discarded.

### SUMMARY

In order to overcome the aforementioned limitations associated with certain nitrogen mustard alkylating oncology agents that consist of solids that require reconstitution prior to administration, a liquid formulation that is generally applicable to such compounds has been developed.

Stable, non-aqueous formulations of nitrogen mustard alkylating oncology agents can be manufactured by solubilizing the compounds in pharmaceutically acceptable organic solvents and stabilizing the resulting solutions by means of inclusion of certain stabilizing agents including a source of chloride ions such as sodium chloride, potassium chloride and one or more of the following: butylated hydroxytoluene (BHT), α-tocopherol, citric acid and ascorbic acid.

Production of such liquid formulations facilitates sterilization by means of filtration and permits inspection of each unit for particulate contamination. Compared to lyophilization, the manufacturing process is simple and requires substantially less time.

Preparations thus made exhibit excellent stability at room temperature and may be stored for protracted periods without the formation of objectionable levels of impurities. Such formulations do not require reconstitution prior to administration, thus greatly simplifying administration procedures.

### DETAILED DESCRIPTION

Embodiments of the invention are directed to stable liquid formulations of the nitrogen mustards. In an embodiment, the nitrogen mustards are selected from the group consisting of mechlorethamine, cyclophosphamide, ifosfamide, melphalan, chlorambucil, uramustine, and thiotepa, individually or in combination with one another. A preferred aspect of the invention is directed to stabilized liquid formulations of one of melphalan, cyclophosphamide, or chlorambucil.

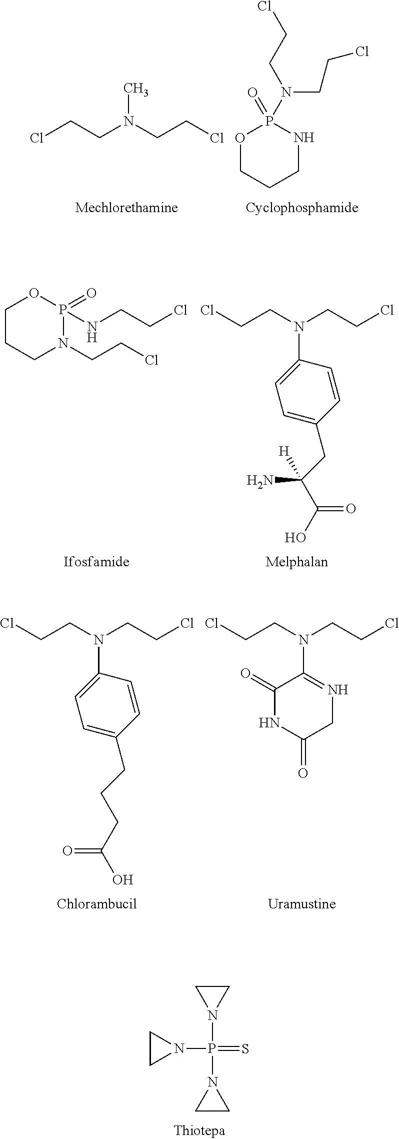

Embodiments of the formulation include the nitrogen mustard at about 0.1% w/w to about 10% w/w of the formulation. In alternative embodiment, the nitrogen mustard comprises about 0.25% w/w to about 2.5% w/w of the formulation.

The stable formulation includes a non-aqueous liquid, an organic acid, and an antioxidant. The non-aqueous liquid includes a first solvent, and may include an optional second solvent.

The non-aqueous liquid may include one or more of the following pharmaceutically acceptable solvents: a glycol, such a polyethylene glycol (PEG), propylene glycol, and a polypropylene glycol (PPG), a diol such as a straight chain, branched, or cyclic aliphatic diol, a triol, such as a straight chain, branched, or cyclic aliphatic triol, a polyoxyethylene ether, and a polyethylene glycol ether.

In an embodiment, the PEG has a molecular weight ranging from about 100 g/mol to about 2,500 g/mol. In an alternative embodiment, the PEG has a molecular weight ranging from between about 300 g/mol to about 1000 g/mol. In a preferred embodiment, the PEG has an average molecular weight ranging from between about 400 g/mol to about 800 g/mol. Exemplary PEGs include PEG-400 having an average molecular weight of about 400 g/mol, PEG-600 having an average molecular weight of about 600 g/mol, and PEG-800 having an average molecular weight of around 800 g/mol.

The polypropylene glycol (PPG) may have a molecular weight ranging from about 200 g/mol to about 2500 g/mol. In a preferred embodiment, the PEG has an average molecular weight ranging from between about 250 g/mol to about 700 g/mol. Exemplary poly propylene glycols include PPG-250 having an average molecular weight of about 250 g/mol, PPG-425 having an average molecular weight of about 425 g/mol, and PPG-700 having an average molecular weight of about 700 g/mol.

In embodiments including a diol solvent, the aliphatic diol may have a general formula of HO—[CH₂]ₙ—OH where n=2-20 and their positional isomers. The aliphatic diol may have either a straight chain or a branched chain and have 2-20 carbon atoms. Exemplary suitable aliphatic diols include 1,2-propane diol, 1,3-propane diol, 1,4-butane diol, 1,5-pentane diol and its higher aliphatic homologs and their positional isomers. The aliphatic cyclic diol may have from 5 to 20 carbon atoms. Exemplary suitable aliphatic cyclic diols include 1,2-cyclopentane diol, 1,2-cyclohexane diol and its higher aliphatic homologs and their positional isomers.

In embodiments including a triol solvent, the aliphatic triol may have from 3 to 20 carbon atoms and may have a straight chain or a branched chain Exemplary suitable aliphatic triols include glycerin (glycerol) and its higher aliphatic homologs and their positional isomers like butane 1,2,3-triol, 1,3,5-pentane triol. The aliphatic cyclic triol may have from 5 to 20 carbon atoms. Exemplary aliphatic cyclic triols include cyclohexane triol, cycloheptanetriol its higher aliphatic homologs and all their positional isomers.

In embodiments including a polyoxyethylene ether, exemplary polyoxyethylene ethers include polysorbate-20 (Tween-20), polysorbate-40 (Tween-40), polysorbate-60 (Tween-60), and polysorbate-80 (Tween-80).

In embodiments including a polyethylene glycol ether, exemplary polyethylene glycol ethers include polyethoxylated castor oil, such as Cremophor® and other poly ethers in that class.

The non-aqueous liquid is generally present in the formulation in a range from about 45% w/w to about 98% w/w of the formulation, and preferably in a range from about 63% w/w to about 98% w/w, and more preferably in a range from about 70% w/w to about 98% w/w. In an embodiment, the first pharmaceutically acceptable non-aqueous liquid is a first solvent with the first solvent present in a range from about 63% w/w to about 98% w/w of the formulation and preferably in a range from about 85% w/w to about 98% w/w. Some nitrogen mustards may not be fully soluble in a neat non-aqueous liquid (i.e., in a first solvent only) and, thus may require a second solvent to enhance the solubility. However, not all solvents are appropriate for this application. Solvents like ethanol or other polarprotic solvents are capable of nucleophilic attack on the carbon containing the chlorine atom in the mustard moiety which will lead to the formation of degradation products and therefore should be avoided or should be kept to a minimum.

Embodiments of non-aqueous liquid may utilize a first solvent individually or in combination with one another, i.e., include at least a second solvent. For example, in one embodiment, the non-aqueous liquid is a first solvent that is PEG-400. In another embodiment, the non-aqueous liquid is a combination of a first solvent, such as PEG-400, and a second solvent, such as propylene glycol. In addition to the solvents described above for use in the non-aqueous liquid, dimethyl acetamide is also a suitable pharmaceutically acceptable second solvent. Thus, in some embodiments, the preferred second solvents include dimethyl acetamide, propylene glycol, glycerin, polysorbate 20, polysorbate 40, polysorbate 80, and Cremophor®.

Some second solvents provide an additional benefit of functioning as a freeze point depressant for the first solvent. For example, polyethylene glycol freezes at around 4 degrees Celsius. The addition of a second solvent, such a propylene glycol or glycerin, decreases the temperature at which polyethylene glycol freezes. The freeze point depressant effects of the second solvent allows the liquid formulations to be stored between 2 and 8 degrees Celsius without having to be concerned about the nitrogen mustard being exposed to one or more freeze thaw cycles. Freeze thaw cycles should be avoided because they can trigger particulate matter that is not desirable in an injectable drug product and also result in the degradation of the nitrogen mustard.

In embodiments wherein the non-aqueous liquid includes a second solvent, the second solvent is present in a range from about 5% w/w to about 55% w/w of the formulation. In another embodiment, the second solvent is present in a range from about 5% w/w to about 40% w/w of the formulation. In another embodiment, the second solvent may be present in a range from about 5% w/w to about 35% w/w of the formulation. In another embodiment, the second solvent may be present in a range from about 10% w/w to about 30% w/w of the formulation. The balance of the non-aqueous liquid may be the first solvent or additional solvents.

In an embodiment, glycerin is the second solvent and is present in a range between about 5% w/w to about 40% w/w of the formulation and preferably in a range from about 10% w/w to about 30% w/w. In another embodiment, propylene glycol is the second solvent and is present in a range between about 5% w/w to about 55% w/w, and preferably in a range from about 15% w/w to about 20% w/w. In another preferred embodiment, dimethyl acetamide is the second solvent and is present in a range from about 5% w/w to about 35% w/w of the formulation, and preferably in a range from about 25% w/w to about 30% w/w.

The pH of the non-aqueous liquid plays a crucial role in the stability of the nitrogen mustard formulation. Protonation of the nitrogen in the mustard moiety avoids the formation of an aziridine ring, which is highly unstable and can result in unacceptable levels of degradation of the nitrogen mustard. An acidic pH is required to maintain the protonated state of the nitrogen in the mustard moiety. In an embodiment, the pH of the formulation is in a range between about pH 0.5 to about pH 6. In another embodiment, the pH is in a range between about pH 1.0 to about pH 4.5. In another embodiment, the pH is in a range between about pH 2.0 to about pH 4.0.

Pharmaceutically acceptable organic acids are used to adjust the pH of the non-aqueous liquid to a level sufficient to maintain the nitrogen in the mustard moiety in a protonated state. The organic acids are present in the formulation at a concentration sufficient to result in a pH within the desired range. Exemplary embodiments of the formulation may include between about 0.02% w/w to about 1.5% w/w organic acid or about 0.5% w/w organic acid.

Exemplary organic acids include citric acid, ascorbic acid, acetic acid, propionic acid, lactic acid, tartaric acid, fumaric acid, succinic acid, oxalic acid, maleic acid, adipic acid, hydroxy acids, alpha hydroxy acids and combinations thereof. In some embodiments, a buffer is used to maintain the desired pH range. In such embodiments, the strength of the buffer and the ionic strength of the non-aqueous liquid play a role in the stability of the formulation. Exemplary buffers include phosphate, citrate, acetate, formate salts and combinations thereof. In an alternative embodiment, inorganic acids, such as hydrochloric acid, phosphoric acid, and amines like an ethanolamine, are used to adjust and buffer the pH of the non-aqueous liquid.

Nitrogen mustards are typically degraded by the nucleophilic attack by water and other solvents like ethanol on the aziridine ring and also by direct attack on the carbon containing the chlorine atom. One can decrease the formation of these degradants by employing chloride ion as a nucleophile (Common Ion Effect) by addition of chloride ion to the formulation (sodium chloride, potassium chloride or any other source of chloride ion that is sufficiently soluble in the chosen formulation solvent). If sufficient chloride ions are present in the formulation, they will compete with other nucleophiles and hinder the degradation of the nitrogen mustard. If chloride ion attacks the aziridine ring, it will tend to reform the original nitrogen mustard moiety as opposed to resulting in the degradation of the nitrogen mustard (See Scheme below).

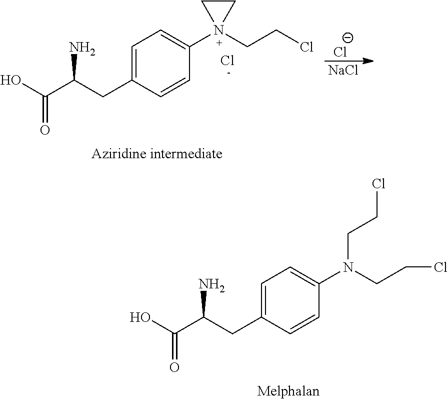

In an embodiment of the invention, the source of chloride ions is present in a range of about 0.01% w/w to about 15% w/w of the formulation. In a preferred embodiment, the source of chloride ions is present in a range of about 0.01% w/w to about 1% w/w of the formulation. In another preferred embodiment, the source of chloride ions is present in a range of about 0.05% w/w to about 0.1% w/w of the formulation.

Nitrogen mustards are also prone to photodegradation and readily degrade when exposed to air or peroxides like hydrogen peroxide by a free radical mechanism. Oxidative degradation can be prevented by using radical scavengers and anti-oxidants. Thus, embodiments of the invention include an effective amount of an anti-oxidant such as butylated hydroxytoluene, α-tocopherol, citric acid, ascorbic acid and combinations thereof. The organic acids may serve the dual functions by also contributing to maintaining the acidic pH of the formulation. The antioxidant may be present at a range of about 0.01% w/w to about 10% w/w of the formulation. In a preferred embodiment, the antioxidant is present at a range of about 0.1% w/w to about 1% w/w of the formulation.

Most of the nitrogen mustards must be stored at between 2-8 degrees Celsius even when in the lyophilized dried power form because these compounds are generally not stable when stored at ambient or elevated temperatures. Embodiments of the invention stabilize nitrogen mustards against degradation at ambient temperatures (e.g., 25 degrees Celsius) and elevated temperatures (e.g., 40 degrees Celsius). Thus, embodiments of the invention confer the additional benefit of allowing nitrogen mustards to be stored at temperatures greater than the typical 2-8 degrees Celsius range, such as at ambient (e.g., about 25 degrees Celsius) or elevated temperatures (e.g., up to about 40 degrees Celsius).

In an aspect of the invention, the nitrogen mustard in the liquid formulation is melphalan. As seen above, melphalan has two nitrogen atoms: the nitrogen in the mustard moiety and the nitrogen in the amino acid moiety. The presence of the mustard moiety nitrogen makes it difficult to develop stable liquid formulations of melphalan due to the formation of highly reactive aziridine ring. The inventive combination of non-aqueous liquid solvent and stabilizing agents significantly stabilizes the liquid formulations of melphalan over a range of temperatures for prolonged periods of time ranging between about 2 weeks to at least 24 months.

Stability of nitrogen mustards may be demonstrated over a range of temperatures. For example, stability may be demonstrated over a range from about 2 degrees Celsius to about 8 degrees Celsius. In these embodiments, the nitrogen mustard is considered stabilized by the formulation if, when stored in this temperature range for at least two weeks, the liquid formulation contains less than 2 percent impurities. In another embodiment, the nitrogen mustard is considered stabilized if the formulation contains less than 5% impurities when stored at this temperature range for at least 3 months, and preferably for at least 6 months, and even more preferably for at least 24 months.

Stability may be demonstrated at 25 degrees Celsius. In an embodiment, a liquid formulation is considered stable if, after storage for 2 weeks at 25 degrees Celsius, the liquid formulation contains less than about 2 percent impurities. In another embodiment, the nitrogen mustard is considered stabilized if the formulation contains less than 5% impurities when stored at this temperature range for at least 3 months, and preferably for at least 6 months, and even more preferably for at least 24 months.

Stability may be demonstrated at 40 degrees Celsius as well. In an embodiment, a liquid formulation is considered stable if, after storage for 2 weeks at 40 degrees Celsius, the liquid formulation contains less than about 2 percent impurities. In another embodiment, the nitrogen mustard is considered stabilized if the formulation contains less than 5% impurities when stored at this temperature range for at least 3 months, and preferably for at least 6 months, and even more preferably for at least 24 months.

The assay and percent impurities may be determined by an UV-HPLC method using a reverse phase C18 column, 5μ particle size, 4.6×250 mm dimension and an HPLC instrument equipped with dual pump, auto sampler and DAD detector.

### Example 1

Five formulations of melphalan were initially evaluated for their ability to stabilize melphalan over a prolonged period of between two months and three months over a range of temperatures from 2 degrees Celsius to about 40 degrees Celsius. The five formulations, listed in Table 1, are compared to stability data collected from a formulation of melphalan in PEG-400 without any stabilizers.

TABLE 1

| | No | Formulation 1 | Formulation 2 | Formulation 3 | Formulation 4 | Formulation 5 |
|---|---|---|---|---|---|---|
| Ingredient | stabilizers | % (w/w) | % (w/w) | % (w/w) | % (w/w) | % (w/w) |
| Melphalan | 1% | 1% | 1% | 1% | 1% | 1% |
| PEG-400 | 99% | 98% | 70% | 85% | 70% | 80% |
| DMA | | | 28.2% | | — | |
| Glycerin | | | | 13.2% | 28.2% | |
| Propylene | | | | | | 18.2% |
| Glycol | | | | | | |
| Anti-oxidant | | 0.07% | 0.07% | 0.07% | 0.07% | 0.07% |
| | | (Tocopherol) | (Tocopherol) | (Tocopherol) | (Tocopherol) | (Tocopherol) |
| Citric acid | | 0.05% | 0.05% | 0.05% | 0.05% | 0.05% |
| Sodium | | 0.7% | 0.7% | 0.7% | 0.7% | 0.7% |
| Chloride | | | | | | |
| Total (%) | | 100% | 100% | 100% | 100% | 100% |

For this example, melphalan was solubilized in the PEG-400 in the presence of the other components as listed in Table 1. Samples of solubilized melphalan (Formulations 1-5) were filled into glass vials, stoppered under nitrogen atmosphere, crimped and stored at 2 degrees Celsius to 8 degrees Celsius, 25 degrees Celsius 60% RH, or 40 degrees Celsius 75% RH for a period of 72 hours for the control and two to three months for the Formulations 1-5. The samples were then examined for the presence of impurities by an UV-HPLC method using a reverse phase C18 column.

As seen in Table 2, at each of the temperatures tested, the non-stabilized formulations of melphalan solubilized in PEG-400, significant impurities were detected after just 72 hours. The 25 degrees Celsius 60% RH, and 40 degrees Celsius 75% RH PEG only formulations were out of specification after just 72 hours and higher levels of impurities were observed in the 2-8 degrees Celsius PEG only formulations.

TABLE 2

| | Impurity-RT | RRT | % Impurity |
|---|---|---|---|
| 25° C., 72 hrs | 6.627 | 0.341 | 9.47 |
| | 8.653 | 0.445 | 0.01 |
| | 14.34 | 0.738 | 0.09 |
| | 19.427 | 1.000 | |
| | 26.527 | 1.365 | 0.08 |
| | 33.453 | 1.722 | 0.12 |
| | | | Total: 9.76% |
| 2-8° C., 72 hrs | 6.627 | 0.341 | 0.14 |
| | 8.613 | 0.444 | 0.01 |
| | 14.347 | 0.739 | 0.12 |
| | 19.42 | 1.000 | |
| | 26.527 | 1.366 | 0.08 |
| | 33.46 | 1.723 | 0.11 |
| | | | Total: 0.46% |
| 40° C., 72 hrs | 6.627 | 0.341 | 16.63 |
| | 8.64 | 0.445 | 0.01 |
| | 13.24 | 0.682 | 0.01 |
| | 14.34 | 0.738 | 0.08 |
| | 18.427 | 0.949 | 0.01 |
| | 19.427 | 1.000 | |
| | 21.36 | 1.100 | 0.01 |
| | 23.947 | 1.233 | 0.03 |
| | 24.953 | 1.284 | 0.01 |
| | 25.673 | 1.322 | 0.04 |
| | 26.26 | 1.352 | 0.06 |
| | 26.527 | 1.365 | 0.06 |
| | 26.8 | 1.380 | 0.13 |
| | 27.247 | 1.403 | 0.17 |
| | 27.633 | 1.422 | 0.15 |
| | 28.013 | 1.442 | 0.12 |
| | 28.407 | 1.462 | 0.08 |
| | 28.787 | 1.482 | 0.05 |
| | 29.2 | 1.503 | 0.02 |
| | 33.453 | 1.722 | 0.11 |
| | | | Total: 17.79% |

As seen in Table 3 below, the presence of stabilizer significantly improved the stability of solubilized melphalan across all temperatures tested over a period of 1, 2, 3, or 11 months.

TABLE 3
Table 3 - Melphalan

| | Time Zero | | 1 Month | | 2 Months | | 3 Months | | 11 Months | |
|---|---|---|---|---|---|---|---|---|---|---|
| | | % Related | | % Related | | % Related | | % Related | | % Related |
| Formulation # | % Assay | Substances | % Assay | Substances | % Assay | Substances | % Assay | Substances | % Assay | Substances |

Storage Condition: 2-8° C.

| | | | | | | | | | | |
|---|---|---|---|---|---|---|---|---|---|---|
| Formulation # 1 | 102.5 | 0.03 | 101.7 | 0.02 | 104.6 | 0.02 | 101.3 | 0.05 | 102.5 | 0.03 |
| Formulation # 2 | 103.4 | 0.04 | 100.5 | 0.02 | 100.9 | 0.03 | 101.1 | 0.02 | 103.4 | 0.04 |
| Formulation # 3 | 103.6 | 0.03 | 103.4 | 0.02 | 103.4 | 0.03 | 101.6 | 0.04 | 103.6 | 0.03 |
| Formulation # 4 | 103.8 | 0.04 | 102.5 | 0.02 | 103.7 | 0.02 | 103.9 | 0.04 | 103.8 | 0.04 |
| Formulation # 5 | 103.1 | 0.03 | 101.8 | 0.02 | 104.2 | 0.03 | 103.8 | 0.02 | 106.1 | 0.03 |

Storage Condition: 25° C./60% RH

| | | | | | | | | |
|---|---|---|---|---|---|---|---|---|
| Formulation # 1 | 102.5 | 0.03 | 100.5 | 0.02 | 103.0 | 0.05 | 100.7 | 0.08 |
| Formulation # 2 | 103.4 | 0.04 | 102.8 | 0.02 | 100.2 | 0.04 | 98.3 | 0.06 |
| Formulation # 3 | 103.6 | 0.03 | 101.0 | 0.03 | 103.0 | 0.06 | 100.3 | 0.07 |
| Formulation # 4 | 103.8 | 0.04 | 102.2 | 0.03 | 102.7 | 0.07 | 101.0 | 0.10 |
| Formulation # 5 | 103.1 | 0.03 | 101.5 | 0.03 | 99.8 | 0.05 | 101.1 | 0.06 |

Storage Condition: 40° C./75% RH

| | | | | | | |
|---|---|---|---|---|---|---|
| Formulation # 1 | 102.5 | 0.03 | 98.9 | 0.14 | 96.8 | 0.27 |
| Formulation # 3 | 103.6 | 0.03 | 100.9 | 0.18 | 96.5 | 0.36 |
| Formulation # 4 | 103.8 | 0.04 | 98.8 | 0.32 | 92.7 | 0.70 |
| Formulation # 5 | 103.1 | 0.03 | 101.0 | 0.14 | 97.1 | 0.31 |

As seen in Table 4 below, the presence of stabilizer significantly improved the stability of solubilized melphalan after 11 months at 2-8 degrees Celsius followed by three months at 25 degrees Celsius totally over a period of about 14 months.

TABLE 4
Table 4 - Melphalan
Stability data ~14 months data: 11 months at 2-8° C. and 3 months at 25° C.

| | Time Zero | | 1 Month | | 2 Months | | 3 Months | |
|---|---|---|---|---|---|---|---|---|
| | % | % Related | % | % Related | % | % Related | % | % Related |
| Formulation | Assay | Substances | Assay | Substances | Assay | Substances | Assay | Substances |
| Formulation 1 | 102.5 | 0.03 | 100.8 | 0.05 | 98.1 | 0.04 | 97.9 | 0.04 |
| Formulation 2 | 103.4 | 0.04 | 96.7 | 0.03 | *94.6 | 0.05 | *92.6 | 0.09 |
| Formulation 3 | 103.6 | 0.03 | 101.2 | 0.04 | 99.0 | 0.04 | 100.4 | 0.07 |
| Formulation 4 | 103.8 | 0.04 | 101.7 | 0.05 | 98.9 | 0.07 | 100.0 | 0.09 |
| Formulation 5 | 103.1 | 0.03 | 99.8 | 0.03 | 97.0 | 0.03 | 97.2 | 0.05 |

Further, as mentioned above, most nitrogen mustards are generally not stable when stored at 25 degrees Celsius. Thus, nitrogen mustard typically must be stored at between 2-8 degrees Celsius even when in the lyophilized dried power form. The data presented herein demonstrate the unexpected result that embodiments of the invention demonstrated in Formulations 1-5 can stabilize melphalan when stored for a prolonged period of time at 25 degrees Celsius and 40 degrees Celsius.

### Example 2

For this example, melphalan was prepared as described above with respect to Formulations 1, 4, and 5 for further analysis. The solubilized melphalan was stored at 2 degrees Celsius to 8 degrees Celsius, 25 degrees Celsius 60% RH, or 40 degrees Celsius 75% RH. After a period of 2 weeks, two months, three months, and six months, the samples were analyzed for assay and for the presence of impurities by an UV-HPLC method using a reverse phase C18 column, as described above.

As seen in Table 5 below, the presence of stabilizer improved the stability of solubilized melphalan across all temperatures tested over a period of 2 weeks or 1 month.

As seen in Tables 5 and 6 below, the presence of stabilizers used in Formulations 1, 4, and 5 improved the stability of solubilized melphalan across all temperatures tested over a period of 2 weeks to 6 months.

These data demonstrate the robustness of embodiments of the invention and confirm the ability of Formulations 1, 4, and 5 to stabilize melphalan over a range of temperatures and for prolonged periods of time.

TABLE 5
Table 5 - Melphalan

| | Time Zero | | 2 Weeks | | 1 Month | |
|---|---|---|---|---|---|---|
| | | % Related | % | % Related | % | % Related |
| Formulation # | Assay | Substances | Assay | Substances | Assay | Substances |

2-8° C.

| | | | | | | |
|---|---|---|---|---|---|---|
| Formulation # 1 | 105.5 | 0.02 | 102.2 | 0.02 | 102.3 | 0.02 |
| Formulation # 4 | 102.1 | 0.02 | 100.2 | 0.02 | 100.6 | 0.02 |
| Formulation # 5 | 105.5 | 0.02 | 100.7 | 0.02 | 101.7 | 0.02 |

No impurities to report at both time points

25° C./60% RH

| | | | | |
|---|---|---|---|---|
| Formulation # 1 | 99.5 | 0.02 | 100.9 | 0.03 |
| Formulation # 4 | 99.1 | 0.03 | 102.7 | 0.05 |
| Formulation # 5 | 100.1 | 0.02 | 101.2 | 0.03 |

No impurities to report at both time points

40° C./75% RH

| | | | | |
|---|---|---|---|---|
| Formulation # 1 | 101.3 | 0.06 | 98.5 | 0.12 |
| Formulation # 4 | 98.3 | 0.14 | 96.7 | *0.41 |
| Formulation # 5 | 98.9 | 0.05 | 100.1 | 0.16 |

No impurities to report at 2 weeks. No impurities to report at 1 month for Formulation #1
and #5.
*Two impurities above the reporting threshold of 0.05%.

It is noted that in Table 6, Formulation 4 had an unusually low assay value when stored at 2-8 degrees Celsius and 40 degrees Celsius, but not at 25 degrees Celsius. No other sample stored at 2-8 degrees Celsius that has been assayed thus far has tested below 98%. It is suspected that the low assay value for Formulation 4 at 2-8 degrees Celsius could be due to sample preparation or injection error. After 3 months at 2-8 degrees Celsius, this sample assayed 101.4%, clearly indicating that the unusual low assay obtained during 2 month time point was either due to sample preparation or injection error rather than a formulation issue. It is also noted that Formulation 4 has a decrease assay value and increased impurity value compared to the other samples stored at 40 degrees Celsius. These data indicate that while this formulation provides improved stability relative to the non-stabilized formulation (Table 2), Formulation 4 might not provide as much stability at elevated temperatures.

TABLE 6
Table 6 - Melphalan

| | Time Zero | | 2 Months | | 3 Months | | 6 Months | |
|---|---|---|---|---|---|---|---|---|
| | % | % Related | % | % Related | % | % Related | % | % Related |
| Formulation | Assay | Substances | Assay | Substances | Assay | Substances | Assay | Substances |

Storage Condition: 2-8° C.

| | | | | | | | | |
|---|---|---|---|---|---|---|---|---|
| Formulation 1 | 105.5 | 0.02 | 96.2 | 0.02 | 102.0 | 0.02 | 102.6 | 0.03 |
| Formulation 4 | 102.1 | 0.02 | 86.4 | 0.02 | 101.4 | 0.02 | 102.8 | 0.03 |
| Formulation 5 | 105.5 | 0.02 | 96.6 | 0.02 | 101.7 | 0.02 | 101.3 | 0.03 |

Storage Condition: 25° C./60% RH

| | | | | | | | | |
|---|---|---|---|---|---|---|---|---|
| Formulation 1 | 105.5 | 0.02 | 98.2 | 0.04 | 90.9 | 0.05 | 100.3 | 0.1 |
| Formulation 4 | 102.1 | 0.02 | 99.0 | 0.09 | 98.5 | 0.14 | 97.5 | 0.31 |
| Formulation 5 | 105.5 | 0.02 | 98.3 | 0.03 | 100.4 | 0.06 | 99.6 | 0.11 |

Storage Condition: 40° C./75% RH

| | | | | | | | | |
|---|---|---|---|---|---|---|---|---|
| Formulation 1 | 105.5 | 0.02 | 100.7 | 0.34 | 94.2 | 0.52 | 83.1 | 1.09 |
| Formulation 4 | 102.1 | 0.02 | 89.9 | 1.13 | 82.5 | 1.61 | 57.9 | 3.53 |
| Formulation 5 | 105.5 | 0.02 | 98.9 | 0.38 | 94.9 | 0.59 | 83.3 | 1.51 |

### Example 3

This example demonstrates the ability of Formulations 1, 4, and 5 from example 1 to stabilize cyclophosphamide and further demonstrates the robustness of these formulations to stabilize nitrogen mustards. For these experiments, cyclophosphamide was prepared as described above with respect to Formulations 1, 4, and 5 in example 1, with the exception being that cyclophosphamide was used in place of melphalan. The solubilized cyclophosphamide was stored at 2 degrees Celsius to 8 degrees Celsius, 25 degrees Celsius 60% RH, or 40 degrees Celsius 75% RH. After a period of 2 weeks, 1 month, 2 months, 3 months, or 6 months, the samples were assayed for the presence of impurities by an UV-HPLC method using a reverse phase C18 column, as described above.

Cyclophosphamide is sensitive to heat and is highly susceptible to degradation. The USP recommended storage temperature for lyophilized cyclophosphamide is less than 25 degrees Celsius. Table 7 demonstrates that Formulations 1, 4, and 5 provide satisfactory stability for cyclophosphamide when stored at 2-8 degrees Celsius for 3 months. Formulation 1 provides satisfactory stability when stored at 25 degrees Celsius for 1 month. Formulation 2 provides satisfactory stability when stored at 25 degrees Celsius for 2 weeks.

TABLE 7
Table 7 - CYCLOPHOSPHAMIDE

| | | | | | |
|---|---|---|---|---|---|
| Formulation | Time Zero | 2 Weeks | 1 Month | 3 Months | 6 Months |
| # | % Assay | % Assay | % Assay | % Assay | % Assay |

2-8° C.

| | | | | | |
|---|---|---|---|---|---|
| Formulation | 99.7 | 100.4 | 98.8 | 100.1 | 87.4 |
| # 1 | | | | | |
| Formulation | 102.0 | 96.8 | 95.5 | 97.4 | 84.8 |
| # 4 | | | | | |
| Formulation | 101.3 | 100.0 | 100.3 | 99.5 | 88.3 |
| # 5 | | | | | |

25° C./60% RH

| | | | | |
|---|---|---|---|---|
| Formulation | 95.2 | 95.8 | 81.2 | 63.7 |
| # 1 | | | | |
| Formulation | 92.1 | 87.4 | 72.2 | 53.4 |
| # 4 | | | | |
| Formulation | 99.3 | 88.1 | 75.5 | 68.4 |
| # 5 | | | | |

40° C./75% RH

| | | | | |
|---|---|---|---|---|
| Formulation | 83.4 | 61.0 | 21.8 | 2.6 |
| # 1 | | | | |
| Formulation | 74.7 | 48.4 | 13.3 | 2.2 |
| # 4 | | | | |
| Formulation | 87.1 | 60.1 | 25.2 | 4.4 |
| # 5 | | | | |

Note:
Cyclophosphamide is sensitive to heat.
The USP recommended storage for Cyclophosphamide for injection (Lyophilized cake) is <25° C.

### Example 4

This example demonstrates the ability of Formulations 1, 4, and 5 from example 1 to stabilize chlorambucil and provide another example of these formulations stabilizing a nitrogen mustard formulation. For these experiments, chlorambucil was prepared as described above with respect to Formulations 1, 4, and 5 in Example 1, with the exception being that chlorambucil was used in place of melphalan. The solubilized chlorambucil was stored at 2 degrees Celsius to 8 degrees Celsius, 25 degrees Celsius 60% RH, or 40 degrees Celsius 75% RH. After a period of 1 month or 3 months, the samples were assayed for the presence of impurities by an UV-HPLC method using a reverse phase C18 column, as described above.

Table 8 below demonstrates that Formulations 1, 4, and 5 provide satisfactory stability to chlorambucil when stored at 2-8 degrees Celsius or 25 degrees Celsius for 3 months.

TABLE 8
Table 8 - CHLORAMBUCIL

| | | | |
|---|---|---|---|
| | Time Zero | 1 Month | 3 Months |
| Formulation # | Assay | % Assay | % Assay |

2-8° C.

| | | | |
|---|---|---|---|
| Formulation # 1 | 103.8 | 101.3 | 97.6 |
| Formulation # 4 | 107.6 | 114.7 | 109.7 |
| Formulation # 5 | 102.2 | 97.8 | 96.4 |

25° C./60% RH

| | | |
|---|---|---|
| Formulation # 1 | 96.1 | 97.4 |
| Formulation # 4 | 102.7 | 100.1 |
| Formulation # 5 | 96.9 | 95.4 |

40° C./75% RH

| | | |
|---|---|---|
| Formulation # 1 | 92.2 | 80.6 |
| Formulation # 4 | 62.2 | 12.4 |
| Formulation # 5 | 88.6 | 55.0 |

Note:
Chlorambucil is sensitive to heat.
The USP recommended storage is <25° C.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A stable liquid formulation of a nitrogen mustard comprising:
a nitrogen mustard in an amount of about 0.1% w/w to about 10% w/w of the formulation, wherein the nitrogen mustard is selected from the group consisting of mechlorethamine, cyclophosphamide, ifosfamide, melphalan, chlorambucil, uramustine, thiotepa and combinations thereof;
a non-aqueous liquid in an amount of about 45% w/w to about 98% w/w of the formulation, the non-aqueous liquid comprising a first solvent;
an antioxidant in an amount of about 0.01% w/w to about 1% w/w of the formulation;
an organic acid in an amount of about 0.02% w/w to about 1.5% w/w of the formulation; and
a source of chloride ions in an amount of about 0.05% w/w to about 15% w/w of the formulation.

2. The composition of claim 1 wherein the nitrogen mustard is melphalan.

3. The composition of claim 1 wherein the nitrogen mustard is chlorambucil.

4. The composition of claim 1 wherein the nitrogen mustard is cyclophosphamide.

5. The composition of claim 1 wherein the first solvent is a polyethylene glycol.

6. The composition of claim 5 wherein the polyethylene glycol is about 63% w/w to about 98% w/w of the formulation.

7. The composition of claim 5 polyethylene glycol is selected from the group consisting essentially of PEG-400, PEG-600, and PEG-800.

8. The composition of claim 1 wherein the non-aqueous liquid further includes a second solvent in an amount of about 5% w/w to about 55% w/w of the formulation.

9. The composition of claim 8 wherein the second solvent is selected from the group consisting essentially of propylene glycol, glycerin, dimethyl acetamide, polysorbate-20, polysorbate-40, polysorbate-80, a polyethoxylated castor oil, and combinations thereof.

10. The composition of one of claim 1 wherein the antioxidant is tocopherol.

11. The composition of claim 10 wherein the tocopherol is about 0.01% w/w to about 10% w/w of the formulation.

12. The composition of one of claim 1 wherein the organic acid is citric acid.

13. The composition of claim 12 wherein the citric acid is about 0.05% of the formulation.

14. The composition of one of claim 1 wherein the source of chloride ions is sodium chloride.

15. The composition of claim 14 wherein the sodium chloride is about 0.01% w/w to about 15% w/w of the formulation.

16. A stable liquid formulation of a nitrogen mustard comprising:
a nitrogen mustard in an amount of about 0.1% w/w to about 10% w/w of the formulation, wherein the nitrogen mustard is selected from the group consisting of mechlorethamine, cyclophosphamide, ifosfamide, melphalan, chlorambucil, uramustine, thiotepa and combinations thereof;
a non-aqueous liquid in an amount of about 45% w/w to about 98% w/w of the formulation, the non-aqueous liquid comprising a first solvent;
an organic acid in an amount sufficient to maintain the nitrogen mustard in a protonated state and to maintain the pH of the formulation;
an antioxidant; and
a source of chloride ions,
wherein the antioxidant and the source of chloride ions are present, in combination, at a concentration effective to prevent the formation of greater than 5% impurities when the formulation is stored at about 25 degrees Celsius for 2 weeks.

17. The composition of claim 16 wherein the antioxidant and the source of chloride ions are provided, in combination, at a concentration effective to prevent the formation of greater than 5% impurities when the formulation is stored at about 25 degrees Celsius for 3 months.

18. A method of improving the stability of a nitrogen mustard comprising:
mixing a nitrogen mustard selected from the group consisting of mechlorethamine, cyclophosphamide, ifosfamide, melphalan, chlorambucil, uramustine, thiotepa and combinations thereof with a non-aqueous liquid in an amount of about 45% w/w to about 98% w/w of the formulation, the non-aqueous liquid comprising a first solvent,
an organic acid in an amount sufficient to maintain the nitrogen mustard in a protonated state and to maintain the pH of the formulation;
an antioxidant; and
a source of chloride ions,
wherein the antioxidant and the source of chloride ions are provided, in combination, at a concentration effective to prevent the formation of greater than 5% impurities when the formulation is stored at about 25 degrees Celsius for 2 weeks.

19. The method of claim 18 further comprising sealing the mixture of claim 18 in a container.

20. The method of claim 18 further comprising storing the mixture in a container at a temperature in a range from about 2 degrees Celsius to about 40 degrees Celsius.
